(19) United States
(12) Patent Application Publication
(10) Pub. No.: US 2025/0228268 A1
(43) Pub. Date: Jul. 17, 2025

(54) CAROTENOID-RICH BLACK SOLDIER FLIES AND FEEDING METHOD AND APPLICATION THEREOF

(71) Applicant: BIOFORTE BIOTECHNOLOGY (SHENZHEN) CO., LTD., Guangdong (CN)

(72) Inventors: Wenfeng HU, Guangdong (CN); Minshan CAI, Guangdong (CN); Jianfeng ZHU, Guangdong (CN); Xu PANG, Guangdong (CN); Bin HU, Guangdong (CN); Xueling LI, Guangdong (CN); Meiyan YANG, Guangdong (CN)

(73) Assignee: BIOFORTE BIOTECHNOLOGY (SHENZHEN) CO., LTD., Guangdong (CN)

(21) Appl. No.: 19/014,220

(22) Filed: Jan. 9, 2025

(30) Foreign Application Priority Data
Jan. 12, 2024 (CN) 202410049517.3

(51) Int. Cl. A23K 50/90; A23K 10/20; A23K 10/30

(52) U.S. Cl. CPC A23K 50/90; A23K 10/20; A23K 10/30

(57) ABSTRACT

The present application relates to the field of poultry feed, and more specifically, it relates to carotenoid-rich black soldier flies and their feeding method and application. A method for feeding carotenoid-rich black soldier flies, including the following steps: step 1, configuring feed for black soldier fly: mixing fat, plant-based materials and compound feed evenly to obtain configured feed for black soldier fly; where the plant-based materials include at least one of carrots, tomato powder and chili meal; where the fat include one or more types of vegetable oil and animal fat; and step 2, feeding black soldier flies with the configured feed and raising them for 8-12 days to obtain carotenoid-rich black soldier flies. The feeding method of the present application extracts natural plant-type carotenoids with black soldier flies.

### CROSS-REFERENCE TO RELATED APPLICATION

This application claims the priority benefit of China application No. 202410049517.3, filed on Jan. 12, 2024. The entirety of the above-mentioned patent application is hereby incorporated by reference herein and made a part of this specification.

### BACKGROUND

### Technical Field

The present invention relates to a field of poultry feed, in particularly, it relates to carotenoid-rich black soldier flies, and a feeding method and application thereof.

### Description of Related Art

Carotenoids are tetraterpenoid pigments widely distributed in photosynthetic bacteria, some archaea and fungi, algae, plants and animals, which first appeared in ancient bacteria about 3 billion years ago, making them one of the oldest known molecules. As of 2018, approximately 850 types of natural carotenoids have been reported, including about 800 types of lutein and about 50 types of carotene. Carotenoids are widely recognized as having important health-promoting functions, such as Vitamin A Original and antioxidant activity, enhancing the immune system and reducing the risk of degenerative diseases such as cancer, cardiovascular diseases, cataracts and macular degeneration. In addition to their biological properties, carotenoids can also be used in the production of natural colorants, food additives, nutritional foods, health products, pharmaceuticals, cosmetics and bioengineered products.

Carotenoids are widely used as feed additives in poultry farming. Besides serving as precursors to vitamins, these pigments can be deposited in the fur, beak, and egg yolk of poultry, thereby improving the quality of meat and eggs.

The global market size for synthetic food colorants in 2022 is reported to be estimated at 590 million US dollars, while the market for natural food colorants had already reached 1.54 billion US dollars in 2021 and is expected to grow at a compound annual growth rate of 7.4%. The global carotenoids market was valued at approximately 1.165 billion US dollars in 2020 and is expected to reach 1.206 billion US dollars by 2026, with an estimated compound annual growth rate of 0.9% from 2021 to 2026. As the backbone of carotenoid products, about two-thirds of the β-carotene in the market comes from synthetic sources, and the supply of natural β-carotene is far from sufficient. Considering the improvement of people's living standards and the concern for nutrition and health, the market demand for natural carotenoids will continue to increase. Currently, the methods for producing carotenoids mainly include natural extraction, chemical synthesis and microbial fermentation.

Natural extraction mainly involves the use of organic solvents to extract carotenoids from plants or algae through various techniques, including Soxhlet extraction, maceration, microwave-assisted extraction, accelerated solvent extraction, pulsed electric field-assisted extraction, supercritical fluid extraction, enzyme-assisted extraction and such like. However, the content of carotenoids in plants and algae is limited, and the exhaust gases, wastewater, and waste residue produced during the extraction process will cause environmental pollution and other issues. Low extraction efficiency, complex process and low recovery rate ultimately lead to high production costs and the inability to supply natural carotenoids on a large scale at a low price. Therefore, even with the continuous development of extraction technology, it is unable to meet the growing human demand for natural carotenoids.

Chemical synthesis mainly uses different intermediates to synthesize carotenoids through the Witting olefination reaction. This method has been applied in production with low raw material costs, mild reaction conditions, high reaction rates and easy separation of products. However, the double-bonds stereoselectivity is difficult to control, and there are varying degrees of chemical reagent residues, which limit the quality, safety, and usage range of some types of products. Carotenoids synthesized by other chemical methods are more or less subject to issues of impure synthetic products, complex synthesis technology and lack of natural activity.

Microbial fermentation mainly utilizes the biological metabolism of microorganisms such as bacteria, algae and yeast to convert raw materials such as glucose, starch and soybean meal into carotenoids, which has the advantages of non-toxicity, harmless, independent of seasonal, regional and climatic factors, and having a short production cycle, etc. Moreover, the synthesized carotenoids are consistent with natural carotenoids in terms of configuration and activity. However, there are issues such as low yield and strict cultivation conditions and such like, and it is currently limited to laboratory research and not suitable for large-scale industrial production.

Chemical synthesis usually requires the use of a large amount of organic solvents, and the synthesized carotenoids are impure; carotenoids produced by natural extraction and microbial fermentation processes are easily oxidized and degraded by light and heat, where degraded carotenoids applied in poultry feed cannot effectively improve the quality of poultry meat and eggs. Therefore, there is still room for improvement.

### SUMMARY

The present invention provides a carotenoid-rich black soldier fly and its feeding method and application in order to address the aforementioned issues.

A first aspect of the present invention is to provide a feeding method for carotenoid-rich black soldier flies, employing the following technical solutions:

A feeding method for carotenoid-rich black soldier flies, including the following steps:
Step 1, configuring feed for black soldier fly:
mixing fat, plant-based materials and compound feed evenly to obtain configured feed for black soldier fly;
where the plant-based materials include at least one of carrots, tomato powder and chili meal;
where the fat includes one or more types of vegetable oil and animal fat; and
Step 2, feeding black soldier flies with the configured feed and raising them for 8-12 days to obtain carotenoid-rich black soldier flies.

By employing the above method, a new approach to extracting natural plant-type carotenoids using black soldier flies has been pioneered, which significantly improves the production efficiency of carotenoids compared to methods of synthesis, chemical extraction or microbial fermentation.

The feeding method provided in the present invention utilizes the black soldier flies as carriers, consuming a large amount of carotenoid-containing feed for black soldier fly, thereby obtaining carotenoid-rich black soldier flies. Compared to chemical synthesis methods, the black soldier flies in this invention are biological entities, making the extraction method more environmentally friendly; moreover, compared to any other methods of synthetic or microbial fermentation, carotenoids are enriched within the bodies of the black soldier flies, isolated from the air and less prone to oxidation, thus receiving excellent protection. Directly using the black soldier flies specially fed to feed poultry can supplement carotenoids for the later and exert the improving effects of carotenoids on the quality of meat and eggs. Black soldier flies are not only rich in proteins, fats and other biomolecules, but also contain carotenoids deposited in the fur, beak, and egg yolk of poultry, thereby improving the quality of meat and eggs, which are safer, more effective and less costly than artificial color additives.

Furthermore, carotenoids in traditional feed are unstable organic compounds that can be affected by factors such as grain variety, harvest season, climate and storage conditions. In contrast, the insect feed prepared in the present invention is less affected by seasons and climate, has a short cycle, and can be produced in large quantities in a short period, enabling large-scale production.

In actual research and development, it was found that if black soldier flies consume plant-based materials containing carotenoids alone, the bioavailability is relatively low. Therefore, specific fat are further selected to be mixed with plant-based materials and compound feed to prepare feed for black soldier fly.

Black soldier flies are rich in proteins and fats. There is a certain molecular interaction between the protein substances and carotenoids within the black soldier flies, which can enhance the stability of carotenoids. Carotenoids are liposoluble functional pigments, therefore when carotenoids (plant-based materials) and fats are consumed simultaneously, the fats are digested to produce free fatty acids, forming a micellar phase in small intestine, which can enhance the bioavailability of carotenoids.

Preferably, the animal fat is black soldier fly oil.

In the present invention, “black soldier fly oil” refers to the oil obtained by pressing method, which involves drying black soldier fly larvae and then placing them in an oil press to extract, followed by filtering to remove impurities to obtain the black soldier fly oil.

Preferably, the animal fat can also be one or more types of lard, tallow from cattle and chicken fat.

Preferably, the vegetable oil is one or more types of soybean oil, sunflower seed oil, flaxseed oil, palm kernel oil and coconut oil.

Preferably, a weight ratio in the mixture of animal fat to vegetable oil ranges from 1:3 to 1:5.

Preferably, the compound feed includes at least one or a mixture of wheat bran, soybean meal and alfalfa powder.

By adopting the above technical solutions, the compound feed, in combination with plant-based materials and fat, can effectively increase the nutritiousness of the feed for black soldier fly, allowing the black soldier fly larvae to grow more rapidly.

Preferably, the compound feed includes wheat bran, soybean meal and alfalfa powder, with a weight ratio of (4-6):(2-4):(1-3).

By adopting the above technical solutions, further specifying the types and proportion of the compound feed and mixing with plant-based materials and fat, allows the black soldier fly larvae to better absorb nutrients and carotenoids, effectively enhancing the content of carotenoids within the black soldier flies.

Preferably, a weight ratio of plant-based materials to compound feed is 1:(0.5-1.5), based on the weight of plant-based materials.

Preferably, the fat is added in an amount of 1-10% based on a total weight of plant-based materials and compound feed.

Preferably, the fat is added in an amount of 5% based on the total weight of plant-based materials and compound feed.

By adopting the above technical solution, further specifying the dosage relationship among plant-based materials, compound feed and fat. Under said specification, the molecular interaction between protein substances and carotenoids within the black soldier flies are stronger, further improving the stability and the content of carotenoids within the black soldier flies, enabling the black soldier flies to have a richer nutrient composition as feed.

Preferably, when configuring said feed for black soldier fly, fat, plant-based materials, compound feed, and water are mixed uniformly to achieve the feed for black soldier fly with a moisture content of 70-80%.

The addition of water for mixing is to provide a liquid environment that facilitates feeding for black soldier fly larvae.

Preferably, after black soldier fly eggs hatch into larvae, using nursery feed for nurturing the larvae till they grow into 4-6 days aged larvae, and then feeding the larvae said feed for black soldier fly.

Preferably, the hatching conditions for black soldier fly eggs are at a temperature of 28±2° C., a humidity of 65±5% and a hatching time of 2-4 days.

Preferably, after 8-12 days of rearing black soldier fly larvae, the harvested larvae are cleaned and frozen.

Preferably, when feeding black soldier fly larvae with the feed for black soldier fly, if using a plastic box and when the plastic box has a dimension of 18 cm*12 cm*7 cm, 150 larvae are placed in each plastic box.

Preferably, when feeding black soldier fly larvae with the feed for black soldier fly, a feeding environmental temperature is 28±2° C., with a relative humidity of 65±5%, and the feeding is protected from light.

Preferably, before harvesting the black soldier fly larvae that have been fed for 8-12 days, they are fasted for 24 hours to empty their intestines.

Preferably, a feed-larva ratio of the feed for black soldier fly (dry feed) to the black soldier fly larvae is (0.5-1.5)g:1 larva, based on the number of black soldier fly larvae.

By adopting the above technical solution, further defining a specific conditions for feeding black soldier fly larvae and providing better growth conditions for the larvae, which is conducive to increasing the content of carotenoids in the black soldier flies, thus being better utilized by poultry.

A second aspect of the present invention is to provide carotenoid-rich black soldier flies.

A third aspect of the present invention is to provide an application of carotenoid-rich black soldier flies in poultry feed.

The present invention feeds black soldier flies with a special way to extract a large amount of carotenoids inside their bodies, and then feeds these special black soldier flies as feed for poultry, effectively supplementing the nutrition for the later. The method of extracting carotenoids is natural, harmless and with purer and more stable carotenoids, which is able to further improve the quality of poultry meat and eggs, having favorable economic benefits. At the same time, it is known to the person of ordinary skill in this field that endogenous carotenoids have higher stability compared to exogenous carotenoids (without special treatment), so the carotenoids enriched in black soldier flies have better stability than carotenoid supplements fed directly to poultry.

Preferably, the carotenoid-rich black soldier flies are added in the diet in an amount of 5-15%.

In summary, the present invention has the following beneficial effects:
1. The present invention pioneers a new approach of extracting natural plant-type carotenoids using black soldier flies, significantly improving the production efficiency of carotenoids compared to methods of synthesis, chemical extraction or microbial fermentation.
2. The present invention preferably uses specific fat with specific plant-based materials and compound feed to make feed for black soldier fly, feeds black soldier fly larvae, utilizes the protein substances in the black soldier flies to enhance the stability of carotenoids and improves bioavailability of the later.
3. The black soldier flies fed in the present invention can be used as feed for poultry, which are rich in carotenoids, and the carotenoids is performance-stable and not easily oxidized and degraded by light and heat, allowing for greater utilization by poultry, thereby improving the quality of poultry meat and eggs and providing nutrients to the poultry.

### DESCRIPTION OF THE EMBODIMENTS

The present application is described in further detail below in conjunction with embodiments.

Raw materials used in the following embodiments and comparative examples are all commercially available products.

A feeding method for carotenoid-rich black soldier flies, including the following steps:
Step 1, hatching eggs:
placing black soldier fly eggs in a constant-temperature-and-humidity incubator with a temperature of 28±2° C. and a relative humidity of 65±5%, and hatching for 2-4 days. After the eggs hatch out, nursery feed is used for nurturing till the black soldier flies grow into 4-6 days aged larvae.

The hatching temperature may be any value of 26° C., 27° C., 28° C., 29° C., 30° C., etc.

The relative humidity may be any value of 60%, 62%, 65%, 67%, 69%, 70%, 72%, 75%, etc.

The hatching time may be any value of 2 days, 2.5 days, 3 days, 3.5 days, 4 days, etc.

The larvae may be any of 4-day-old larvae, 5-day-old larvae, 6-day-old larvae, etc.

The nursery feed includes wheat bran, soybean meal and alfalfa powder, with a weight ratio of (4-6):(2-4):(1-3). Specifically, the weight ratio of wheat bran, soybean meal and alfalfa powder may be 4:4:2, may be 6:2:2, may be 5:4:1, may be 4:3:3, or any other ratio within these ranges.
Step 2, preparing feed for black soldier fly:
grinding plant-based materials, then adding compound feed and fat, and adding water to mix until uniform.

A feed-larva ratio of the feed for black soldier fly (dry feed) to black soldier fly larvae is (0.5-1.5)g:1 larva. Specifically, the feed-larva ratio of the feed for black soldier fly (dry feed) to black soldier fly larvae may be 0.5 g:1 larva, may be 0.8 g:1 larva, may be 1 g:1 larva, may be 1.2 g:1 larva, may be 1.5 g:1 larva, or any other feed-larva ratio within these ranges.

The compound feed includes wheat bran, soybean meal and alfalfa powder, with a weight ratio of (4-6):(2-4):(1-3). Specifically, the weight ratio of wheat bran, soybean meal and alfalfa powder may be 4:4:2, may be 6:2:2, may be 5:4:1, may be 5:3:2, may be 4:3:3, or any other ratio within these ranges.

The plant-based materials include at least one or more of carrots, tomato powder and chili meal.

A weight ratio of the plant-based materials to the compound feed is 1:(0.5-1.5). Specifically, the weight ratio of the plant-based materials to the compound feed may be 1:0.5, may be 1:0.8, may be 1:1, may be 1:1.2, may be 1:1.5, or any other ratio within these ranges.

The fat includes one or more types of vegetable oil and animal fat.

Specifically, the animal fat may be black soldier fly oil, lard, tallow from cattle or chicken fat; and the vegetable oil may be soybean oil, sunflower seed oil, flaxseed oil, palm kernel oil or coconut oil. It should be noted that animal fat and vegetable oils can be add in a mixture to avoid problems of animal fat solidifying at room temperature. The mixed weight ratio of animal fat to vegetable oil is 1:3-1:5, that is, for every 1 part by weight of animal fat, 3 parts, 4 parts, 5 parts, or any value within the range of 3-5 parts by weight of vegetable oil can be added.

Therein, the black soldier fly oil is produced from dried black soldier fly larvae by pressing and filtering.

It should also be noted that the vegetable oil may not necessarily be added if the animal fat is black soldier fly oil, because the black soldier fly oil does not solidify easily at room temperature (25° C.).

Based on a total weight of plant-based materials and compound feed, the fat is added in an amount of 1-10%. Specifically, the fat may be added in an amount of 1.0%, 1.5%, 2.0%, 2.5%, 3.0%, 3.5%, 4.0%, 4.5%, 5.0%, 5.5%, 6.0%, 6.5%, 7.0%, 7.5%, 8.0%, 8.5%, 9.0%, 9.5%, 10.0%, or any other value within this range.

The amount of water added is not strictly limited, with the moisture content of the feed for black soldier fly being 70-80%. That is, the moisture content may be 70%, 72%, 75%, 78%, 80%, or any other value within this range.

There is no strict limitation on grinding. Grinding is done to prepare the plant-based materials in appropriate size for black soldier fly larvae to consume.

Step 3, placing black soldier fly larvae obtained in Step 1 into the feed for black soldier

fly obtained in Step 2, and placing them in a feed container. After feeding for 8-12 days, collecting carotenoid-rich black soldier fly larvae.

A feeding environmental temperature is 28±2° C., with a relative humidity of 65±5%, and the feeding is protected from light. The feeding environmental temperature may be any value of 26° C., 27° C., 28° C., 29° C.,

30° C., etc.

The relative humidity may be any value of 60%, 62%, 65%, 67%, 69%, 70%, 72%, 75%, etc.

The feed container is not strictly limited. A preferred choice is a container with dimensions of 18 cm*12 cm*7 cm, and 150 larvae are placed in each container.

Days of feeding may be any duration of 8 days, 9 days, 10 days, 11 days, 12 days, etc.

Step 4, fasting black soldier fly larvae for 24 h and emptying their intestines when collecting the larvae in Step 3, and then cleaning and freezing the collected larvae.

The larvae are fasted in order to empty residual feed from the insect's digestive tract.

The larvae are frozen in order to make it more convenient to control the bodies of larvae

and for later consumption by poultry.

### EMBODIMENTS

### Embodiment 1-1

A feeding method for carotenoid-rich black soldier flies, including the following steps:
Step 1, hatching eggs:
   placing black soldier fly eggs in a constant-temperature-and-humidity incubator with a temperature of 28° C. and a relative humidity of 65%, and hatching for 3 days. After the eggs hatched out, nursery feed was used for nurturing till the black soldier flies grew into 6-days aged larvae.

The nursery feed includes wheat bran, soybean meal and alfalfa powder, with a weight ratio of 5:3:2.
Step 2, preparing feed for black soldier fly:
grinding plant-based materials, then adding compound feed and vegetable oil, and adding water to mix until uniform. A feed-larva ratio of the feed for black soldier fly (dry feed) to black soldier fly larvae is 1 g:1 larva.

The plant-based materials are carrots. The vegetable oil is soybean oil. The compound feed includes wheat bran, soybean meal and alfalfa powder, with a weight ratio of 5:3:2.

A weight ratio of the plant-based materials to compound feed is 1:1.

Based on a total weight of plant-based materials and compound feed, the amount of vegetable oil added is 1%.

The moisture content of the feed for black soldier fly is 75%.
Step 3, placing black soldier fly larvae obtained in Step 1 into the feed for black soldier fly obtained in Step 2, and placing them in a feed container. After feeding for 10 days, collecting carotenoid-rich black soldier fly larvae. In this embodiment, the feed for black soldier fly is a one-time feeding, and no additional feed is required during the feeding process.

A feeding environmental temperature is 28° C., with a relative humidity of 65%, and the feeding is protected from light.

The feed container is a plastic box, which has a dimension of 18 cm*12 cm*7 cm, and 150 larvae are placed in each plastic box.
Step 4, separating the larvae from the feed with a sieve, rinsing the obtained larvae with clean water, fasting the larvae for 24 hours to empty residual feed from the insect's digestive tract, and obtaining fasted larvae. Separating molts and feces from the black soldier flies with the sieve, rinsing with clean water and removing surface moisture to obtain carotenoid-rich black soldier flies.

### Embodiment 1-2

A feeding method for carotenoid-rich black soldier flies, differing from Embodiment 1-1 is that the amount of vegetable oil added is 2%.

### Embodiment 1-3

A feeding method for carotenoid-rich black soldier flies, differing from Embodiment 1-1 is that the amount of vegetable oil added is 5%.

### Embodiment 1-4

A feeding method for carotenoid-rich black soldier flies, differing from Embodiment 1-1 is that the amount of vegetable oil added is 10%.

### Embodiment 2-1

A feeding method for carotenoid-rich black soldier flies, differing from Embodiment 1-1 is that the plant-based materials are tomato powder and the vegetable oil is sunflower seed oil.

### Embodiment 2-2

A feeding method for carotenoid-rich black soldier flies, differing from Embodiment 2-1 is that the amount of vegetable oil added is 2%.

### Embodiment 2-3

A feeding method for carotenoid-rich black soldier flies, differing from Embodiment 2-1 is that the amount of vegetable oil added is 5%.

### Embodiment 2-4

A feeding method for carotenoid-rich black soldier flies, differing from Embodiment 2-1 is that the amount of vegetable oil added is 10%.

### Embodiment 3-1

A feeding method for carotenoid-rich black soldier flies, differing from Embodiment 1-1 is that the plant-based materials are chili meal and the vegetable oil is flaxseed oil.

### Embodiment 3-2

A feeding method for carotenoid-rich black soldier flies, differing from Embodiment 3-1 is that the amount of vegetable oil added is 2%.

### Embodiment 3-3

A feeding method for carotenoid-rich black soldier flies, differing from Embodiment 3-1 is that the amount of vegetable oil added is 5%.

### Embodiment 3-4

A feeding method for carotenoid-rich black soldier flies, differing from Embodiment 3-1 is that the amount of vegetable oil added is 10%.

### Embodiment 4-1

A feeding method for carotenoid-rich black soldier flies, differing from Embodiment 1-1 is that the soybean oil is replaced with black soldier fly oil.

### Embodiment 4-2

A feeding method for carotenoid-rich black soldier flies, differing from Embodiment 4-1 is that the amount of black soldier fly oil added is 2%.

### Embodiment 4-3

A feeding method for carotenoid-rich black soldier flies, differing from Embodiment 4-1 is that the amount of black soldier fly oil added is 5%.

### Embodiment 4-4

A feeding method for carotenoid-rich black soldier flies, differing from Embodiment 4-1 is that the amount of black soldier fly oil added is 10%.

### Embodiment 5-1

A feeding method for carotenoid-rich black soldier flies, differing from Embodiment 2-1 is that the sunflower seed oil is replaced with black soldier fly oil.

### Embodiment 5-2

A feeding method for carotenoid-rich black soldier flies, differing from Embodiment 5-1 is that the amount of black soldier fly oil added is 2%.

### Embodiment 5-3

A feeding method for carotenoid-rich black soldier flies, differing from Embodiment 5-1 is that the amount of black soldier fly oil added is 5%.

### Embodiment 5-4

A feeding method for carotenoid-rich black soldier flies, differing from Embodiment 5-1 is that the amount of black soldier fly oil added is 10%.

### Embodiment 6-1

A feeding method for carotenoid-rich black soldier flies, differing from Embodiment 3-1 is that the flaxseed oil is replaced with black soldier fly oil.

### Embodiment 6-2

A feeding method for carotenoid-rich black soldier flies, differing from Embodiment 6-1 is that the amount of black soldier fly oil added is 2%.

### Embodiment 6-3

A feeding method for carotenoid-rich black soldier flies, differing from Embodiment 6-1 is that the amount of black soldier fly oil added is 5%.

### Embodiment 6-4

A feeding method for carotenoid-rich black soldier flies, differing from Embodiment 6-1 is that the amount of black soldier fly oil added is 10%.

### Embodiment 7-1

A feeding method for carotenoid-rich black soldier flies, differing from Embodiment 1-1 is that the soybean oil is replaced with a mixture of lard and soybean oil, and the amount of the mixture of lard and soybean oil added is 1%, and a weight ratio of lard to soybean oil is 1:3.

### Embodiment 7-1-1

A feeding method for carotenoid-rich black soldier flies, differing from Embodiment 7-1 is that the weight ratio of lard to soybean oil is 1:4.

### Embodiment 7-1-2

A feeding method for carotenoid-rich black soldier flies, differing from Embodiment 7-1 is that the weight ratio of lard to soybean oil is 1:5.

### Embodiment 7-2

A feeding method for carotenoid-rich black soldier flies, differing from Embodiment 7-1 is that the amount of the mixture of lard and soybean oil added is 2%.

### Embodiment 7-3

A feeding method for carotenoid-rich black soldier flies, differing from Embodiment 7-1 is that the amount of the mixture of lard and soybean oil added is 5%.

### Embodiment 7-4

A feeding method for carotenoid-rich black soldier flies, differing from Embodiment 7-1 is that the amount of the mixture of lard and soybean oil added is 10%.

### COMPARATIVE EXAMPLES

### Comparative example 1

A feeding method for carotenoid-rich black soldier flies, differing from Embodiment 1-1 is that no vegetable oil is added.

### Comparative example 2

A feeding method for carotenoid-rich black soldier flies, differing from Embodiment 2-1 is that no vegetable oil is added.

### Comparative example 3

A feeding method for carotenoid-rich black soldier flies, differing from Embodiment 3-1 is that no vegetable oil is added.

It should be noted that the vegetable oil in the aforementioned embodiments may also be replaced with palm kernel oil or coconut oil, and the corresponding amount of carotenoids in the bodies of black soldier flies are slightly higher than that of soybean oil, achieving a similar beneficial effect.

### Application Example

An application of carotenoid-rich black soldier flies in poultry feed.

Specifically, black soldier flies obtained and fed by methods of the embodiments was used to feed poultry. The black soldier flies can be fed directly to poultry or mixed with daily ration.

There is no strict limitation on the amount of black soldier flies added to the daily ration, but preferably it is 5-15%. The amount of black soldier flies added to the daily ration may be 5%, 8%, 10%, 12%, 15%, or any other value within this range.

### Application Example 1

An application of carotenoid-rich black soldier flies in poultry feed.

Using the black soldier flies fed according to Embodiment 1-1 to feed Hyland Grey laying hens, and the amount of black soldier flies added to the daily ration is 10%.

### Performance Testing Experiment

1. Determination of carotenoid content in black soldier flies:

Determination of carotenoid content in black soldier fly larvae: 0.5g of finely ground sample was extracted using 10 mL of anhydrous ethanol/acetone/n-hexane extractant (1:1:2, v/v/v; containing 0.1% BHT, w/v), vortexed and shaken for 60 seconds, and then placed in a refrigerator (4° C.) for 18 hours.

Centrifuge tubes were taken from the refrigerator, and then the centrifuge tubes containing samples were vortexed on a shaker for 60 seconds, and then sonicated in a ultrasonic bath for 10 minutes (room temperature, power of 40 W), and then centrifuged at 8000 r/min for 10 minutes.

The supernatant was transferred to a 250 mL round-bottom flask and the precipitate was re-extracted with 10 mL of the extractant.

The two supernatants were combined and evaporated to dryness under vacuum at 40° C. The extract was fully dissolved with the extractant, fixed to 10 mL, a portion of the solution was taken to determine the absorbance at 450 nm on a UV-visible spectrophotometer and the absorbance was recorded in Table 1.

The samples to be tested were the black soldier flies fed by the Embodiments and the

### COMPARATIVE EXAMPLES

TABLE 1
Total Carotenoid Content on a Dry Weight Basis

| | Total Carotenoid | | Total Carotenoid |
|---|---|---|---|
| | Content on a Dry | | Content on a Dry |
| | Weight Basis | | Weight Basis |
| Category | (μg/g) | Category | (μg/g) |
| Comparative | 20.20 ± 0.39 | / | / |
| Example 1 | | | |
| Embodiment 1-1 | 20.48 ± 0.34 | Embodiment 4-1 | 23.27 ± 0.27 |
| Embodiment 1-2 | 21.64 ± 0.45 | Embodiment 4-2 | 24.11 ± 0.34 |
| Embodiment 1-3 | 29.36 ± 0.74 | Embodiment 4-3 | 31.66 ± 0.69 |
| Embodiment 1-4 | 31.21 ± 0.51 | Embodiment 4-4 | 33.70 ± 0.72 |
| Comparative | 19.23 ± 0.69 | / | / |
| Example 2 | | | |
| Embodiment 2-1 | 20.26 ± 0.26 | Embodiment 5-1 | 21.26 ± 0.28 |
| Embodiment 2-2 | 22.26 ± 1.09 | Embodiment 5-2 | 23.89 ± 0.53 |
| Embodiment 2-3 | 23.29 ± 0.38 | Embodiment 5-3 | 26.77 ± 0.65 |
| Embodiment 2-4 | 24.89 ± 0.74 | Embodiment 5-4 | 28.51 ± 0.73 |
| Comparative | 8.89 ± 0.44 | / | / |
| Example 3 | | | |
| Embodiment 3-1 | 9.64 ± 0.26 | Embodiment 6-1 | 11.31 ± 0.17 |
| Embodiment 3-2 | 10.14 ± 0.27 | Embodiment 6-2 | 12.27 ± 0.42 |
| Embodiment 3-3 | 12.09 ± 0.16 | Embodiment 6-3 | 14.66 ± 0.41 |
| Embodiment 3-4 | 14.21 ± 0.37 | Embodiment 6-4 | 16.09 ± 0.21 |
| Embodiment 7-1 | 23.19 ± 0.21 | Embodiment 7-2 | 24.22 ± 0.74 |
| Embodiment 7-1-1 | 22.56 ± 0.34 | Embodiment 7-3 | 31.11 ± 0.45 |
| Embodiment 7-1-2 | 21.87 ± 0.26 | Embodiment 7-4 | 32.87 ± 0.35 |

Based on the comparison of the test data between Embodiment 1 and Comparative Example 1 in Table 1, the carotenoid content in the black soldier flies of Embodiment 1-1, Embodiment 1-2, Embodiment 1-3 and Embodiment 1-4 increased by 1.40%, 7.15%, 45.39% and 54.50% respectively compared to that of Comparative Example 1. This indicates that the combination of vegetable oil (soybean oil) and plant-based materials can indeed improve the stability of carotenoids in black soldier flies. When the amount of soybean oil added is within the range of 1%- 10%, the content of carotenoids in black soldier flies increases with the addition of soybean oil. Based on the comparison of the test data between Embodiment 2 and Comparative Example 2 in Table 1, the carotenoid content in the black soldier flies of Embodiment 2-1, Embodiment 2-2, Embodiment 2-3 and Embodiment 2-4 increased by 5.39%, 15.78%, 21.15% and 29.43% respectively compared to Comparative Example 2. The addition of sunflower seed oil can effectively increase the content of carotenoids in black soldier flies, and the enhancement is more stable when the addition of sunflower seed oil is within the range of 2-10%.

According to the comparison of the test data between Embodiment 3 and Comparative Example 3 in Table 1, the carotenoid content in the black soldier flies of Embodiment 3-1, Embodiment 3-2, Embodiment 3-3 and Embodiment 3-4 increased by 8.44%, 14.10%, 35.96% and 59.84% respectively compared to Comparative Example 3. The addition of flaxseed oil within the range of 1-10% can significantly increase the carotenoid content. Compared with the enhancements in Embodiment 1 and 2, the combination of flaxseed oil and chili meal is relatively stable and the enhancement is significant.

According to the comparison of the test data between Embodiment 4 and Embodiment 1 in Table 1, the carotenoid content in the black soldier flies of Embodiments 4-1, 4-2, 4-3 and 4-4 increased by 13.62%, 11.41%, 7.83% and 7.98% respectively compared to Embodiments 1-1, 1-2, 1-3 and 1-4.

According to the comparison of the test data between Embodiment 5 and Embodiment 2 in Table 1, the carotenoid content in the black soldier flies of Embodiments 5-1, 5-2, 5-3 and 5-4 increased by 4.94%, 7.32%, 14.94% and 14.54% respectively compared to Embodiments 2-1, 2-2, 2-3 and 2-4.

According to the comparison of the test data between Embodiment 6 and Embodiment 3 in Table 1, the carotenoid content in the black soldier flies of Embodiments 6-1, 6-2, 6-3 and 6-4 increased by 17.32%, 21.01%, 21.26% and 13.23% respectively compared to Embodiments 3-1, 3-2, 3-3 and 3-4. The above comparisons show that under the same conditions, black soldier fly oil is superior to vegetable oils (soybean oil, sunflower seed oil, and flaxseed oil) in increasing the carotenoid content in black soldier flies.

According to the comparison of the test data between Embodiment 7 and Embodiment 1 in Table 1, the carotenoid content in the black soldier flies of Embodiments 7-1, 7-2, 7-3 and 7-4 increased by 13.23%, 11.92%, 5.96% and 5.31% respectively compared to Embodiments 1-1, 1-2, 1-3 and 1-4. This indicates that a mixture of animal fat and vegetable oil is superior to single vegetable oil in increasing the carotenoid content in black soldier flies.

According to the comparison of the test data between Embodiment 7-1, Embodiment 7-1-1 and Embodiment 7-1-2 in Table 1, with the same amount of fat added, the more animal fat is included, the more carotenoids are present in the black soldier flies, indicating that animal fat is more effective than vegetable oil in enriching the carotenoid content in black soldier flies.
2. Impact of black soldier fly as the feed to poultry:

A total of 300 Hyland Grey laying hens with similar weight and good health status, during their peak laying period, were selected and randomly divided into 5 treatments. Each treatment was set 4 replicates and 15 hens for each replicate, and a pre-feeding period was 7 days and a regular feeding period was 28 days. Feeding was done regularly, with 115 g of feed for each hen per day.

Group A was a normal group, fed with normal diet; Group B was a regular-black-soldier-fly-larvae group, with 10% regular black soldier fly larvae added to the diet; Group C was a carotenoid-rich-black-soldier-fly-larvae group, with 10% of the carotenoid-rich black soldier flies which were fed according to Embodiment 1-3 (with 5% of vegetable oil) added to the diet; Group D was also a carotenoid-rich-black-soldier-fly-larvae group, with 10% of the carotenoid-rich black soldier flies which were fed according to Embodiment 4-3 (with 5% black soldier fly oil) added to the diet; and Group E was another carotenoid-rich-black-soldier-fly-larvae group, with 10% of the carotenoid-rich black soldier flies which were fed according to Embodiment 7-3 (5% addition of a mixture of lard and soybean oil with a weight ratio of 1:3) added to the diet.

In this test, the normal diet used in Group A included 30% of soybean meal, 1.5% of soybean oil, 8% of mineral meal, 1% of peptide nucleic acid, premix, and rice to supplement the rest to 100%.

The premix included providing per kilogram of normal diet: copper 2400 mg, iron 2000 mg, zinc 3400 mg, manganese 3200 mg, iodine 35 mg, selenium 8 mg, vitamin A acetate 300KIU, vitamin D3 100KIU, vitamin E 440 mg, vitamin K340 mg, vitamin B1 60 mg, vitamin B2 200 mg, vitamin B6 120 mg, vitamin B12 0.6 mg, biotin 4 mg, niacinamide 880 mg, D-pantothenic acid 320 mg, folic acid 24 mg, choline chloride 8000 mg, phytase 1200 mg, methionine 45g, total phosphorus 40g and sodium chloride 60g.

The feeding method for the regular black soldier fly larvae in Group B was as follows:
Step 1, hatching eggs:

Placing black soldier fly eggs in a constant-temperature-and-humidity incubator with a temperature of 28° C. and a relative humidity of 65%, and hatching for 3 days. After the eggs hatched out, nursery feed was used for nurturing till the black soldier flies grew into 6-days aged larvae.

The nursery feed includes wheat bran, soybean meal and alfalfa powder, with a weight ratio of 5:3:2.
Step 2, preparing feed for black soldier fly:

Mixing wheat bran, soybean meal and alfalfa powder in a weight ratio of 5:3:2, then adding water and mixing. The moisture content of the feed for black soldier fly was 75%.

A feed-larva ratio of the feed for black soldier fly (dry feed) to black soldier fly larvae was 1 g:1 larva.
Step 3, placing black soldier fly larvae obtained in Step 1 into the feed for black soldier fly obtained in Step 2, and placing them in a feed container. After feeding for 10 days, collecting black soldier fly larvae. In this test, the feed for black soldier fly was a one-time feeding, and no additional feed was required during the feeding process.

A feeding environmental temperature was 28° C., with a relative humidity of 65%, and the feeding was protected from light.

The feed container was a plastic box, which had a dimension of 18 cm*12 cm*7 cm, and 150 larvae were placed in each plastic box.
Step 4, separating the larvae from the feed with a sieve, rinsing the obtained larvae with

clean water, fasting the larvae for 24 hours to empty residual feed from the insect's digestive tract, and obtaining fasted black soldier flies. Separating molts and feces from the black soldier flies with the sieve, rinsing with clean water and removing surface moisture to obtain regular black soldier flies.

TABLE 2
Egg Production Rate by Different Groups

| | | | | | |
|---|---|---|---|---|---|
| Egg Production | | | | | |
| Rate (%) | A | B | C | D | E |

| | | | | | | |
|---|---|---|---|---|---|---|
| 7 | d | 87.5 | 88.19 | 88.19 | 88.20% | 87.80% |
| 14 | d | 84.03 | 86.11 | 84.72 | 87.01% | 85.22% |
| 21 | d | 88.89 | 90.97 | 90.97 | 90.88% | 90.10% |
| 28 | d | 86.81 | 90.97 | 87.44 | 91.27% | 89.95% |

TABLE 3
Average Egg Weight by Different Groups

| Average Egg | | | | | | |
|---|---|---|---|---|---|---|
| Weight (g/item) | | A | B | C | D | E |
| 7 | d | 66.86 ± 1.25 | 67.02 ± 1.14 | 68.74 ± 2.67 | 69.1 ± 1.37 | 68.89 ± 1.08 |
| 14 | d | 65.80 ± 0.88 | 66.09 ± 1.17 | 68.22 ± 0.42 | 68.77 ± 0.67 | 68.63 ± 0.51 |
| 21 | d | 64.96 ± 0.62 | 66.23 ± 0.76 | 68.37 ± 0.89 | 68.67 ± 0.65 | 68.39 ± 0.61 |
| 28 | d | 64.17 ± 1.00 | 66.50 ± 0.86 | 67.71 ± 0.46 | 68.21 ± 1.12 | 67.89 ± 0.73 |

TABLE 4
Carotenoid Content in Egg Yolk by Different Groups

| Carotenoid Content | | | | | | |
|---|---|---|---|---|---|---|
| in Egg Yolk (μg/g) | | A | B | C | D | E |
| 7 | d | 11.51 ± 0.04 | 8.35 ± 0.23 | 10.13 ± 0.11 | 13.26 ± 0.21 | 12.67 ± 0.19 |
| 14 | d | 16.97 ± 0.39 | 15.53 ± 0.17 | 21.58 ± 0.08 | 23.1 ± 0.15 | 22.08 ± 0.34 |
| 21 | d | 16.74 ± 0.20 | 16.08 ± 0.14 | 22.63 ± 0.04 | 23.67 ± 0.11 | 22.43 ± 0.21 |
| 28 | d | 13.87 ± 0.32 | 14.64 ± 0.05 | 22.39 ± 0.20 | 24.18 ± 0.07 | 23.21 ± 0.16 |

According to the test data in Tables 2-4, the black soldier flies obtained from the feeding method provided in the present invention, when used as feed for laying hens (poultry), can achieve egg production rates comparable to those of a normal diet, while there is a relatively significant improvement in terms of the egg weight, and especially a more significant improvement in terms of carotenoid content in egg yolk. Moreover, in terms of the improvement of the carotenoid content in egg yolk, even with a shorter feeding period, a significant difference can be made compared that of a normal diet and feed, offering good economic benefits.

In summary, the black soldier flies obtained from the feeding method provided in the present invention as poultry feed can improve the production performance and egg quality of poultry.

These specific embodiments are merely explanations of this invention and do not limit the scope of the present invention. Technical personnel in this field, after reading this specification, can make modifications to these embodiments without involving creative contributions, and those are protected by patent laws as long as within the scope of the claims of this invention.

What is claimed is:

1. A feeding method for carotenoid-rich black soldier flies, comprising the following steps:
step 1, configuring feed for black soldier fly:
mixing fat, plant-based materials and compound feed evenly to obtain configured feed for black soldier fly;
where the plant-based materials comprise at least one of carrots, tomato powder and chili meal;
where the fat comprises one or more types of vegetable oil and animal fat; and
step 2, feeding the black soldier flies with the configured feed and raising them for 8-12 days to obtain the carotenoid-rich black soldier flies.

2. The feeding method for carotenoid-rich black soldier flies according to claim 1, wherein the animal fat is black soldier fly oil.

3. The feeding method for carotenoid-rich black soldier flies according to claim 1, wherein the animal fat is one or more types of lard, tallow from cattle and chicken fat.

4. The feeding method for carotenoid-rich black soldier flies according to claim 3, wherein the vegetable oil is one or more types of soybean oil, sunflower seed oil, flaxseed oil, palm kernel oil and coconut oil.

5. The feeding method for carotenoid-rich black soldier flies according to claim 4, wherein the fat is a mixture of the animal fat and the vegetable oil, a weight ratio in the mixture of the animal fat to the vegetable oil is 1:3-1:5.

6. The feeding method for carotenoid-rich black soldier flies according to claim 1, wherein the compound feed comprises at least one or a mixture of wheat bran, soybean meal and alfalfa powder.

7. The feeding method for carotenoid-rich black soldier flies according to claim 6, wherein the compound feed consists of wheat bran, soybean meal and alfalfa powder, with a weight ratio of (4-6):(2-4):(1-3).

8. The feeding method for carotenoid-rich black soldier flies according to claim 1, wherein a weight ratio of the plant-based materials to the compound feed is 1:(0.5-1.5), based on a weight of the plant-based materials.

9. The feeding method for carotenoid-rich black soldier flies according to claim 1, wherein the fat is added in an amount of 1-10% based on a total weight of the plant-based materials and the compound feed.

10. The feeding method for carotenoid-rich black soldier flies according to claim 1, wherein during the step 1, the fat, the plant-based materials, the compound feed, and water are mixed uniformly to achieve the configured feed for black soldier fly with a moisture content of 70-80%.

11. The feeding method for carotenoid-rich black soldier flies according to claim 1, wherein the step 2 further comprises hatching black soldier fly eggs into larvae, using nursery feed for nurturing the larvae till they grow into 4-6 days aged larvae, and then feeding the larvae with the configured feed for black soldier fly.

12. The feeding method for carotenoid-rich black soldier flies according to claim 11, wherein the hatching black soldier fly eggs is at a temperature of 28±2° C. and a humidity of 65±5% with a hatching time of 2-4 days.

13. Carotenoid-rich black soldier flies, obtained by using the feeding method for carotenoid-rich black soldier flies according to claim 1.

14. An application of the carotenoid-rich black soldier flies according to claim 13 in poultry feed.
